(19) United States
(12) Patent Application Publication
(10) Pub. No.: US 2016/0136320 A1
(43) Pub. Date: May 19, 2016

(54) LENS CARE PRODUCT FOR OZONE-BASED CLEANING/DISINFECTING OF CONTACT LENSES

(71) Applicants: Robert Carey TUCKER, Suwanee, GA (US); John Dallas PRUITT, Suwanee, GA (US); Leroy Wainaina MUYA, Duluth, GA (US)

(72) Inventors: Robert Carey TUCKER, Suwanee, GA (US); John Dallas PRUITT, Suwanee, GA (US); Leroy Wainaina MUYA, Duluth, GA (US)

(21) Appl. No.: 14/898,426

(22) PCT Filed: Jun. 24, 2014
(86) PCT No.: PCT/US14/43772

§ 371 Date: Dec. 14, 2015

Related U.S. Application Data
(60) Provisional application No. 61/838,548, filed on Jun. 24, 2013.

(51) Int. Cl. A61L 12/12

(52) U.S. Cl. CPC A61L 12/122

(57) ABSTRACT

The present invention provides an aqueous lens care solution for disinfecting and/or cleaning contact lenses in an ozone-based lens care system. The lens care solution of the invention is a hypotonic solution, has an osmolality at about 25° C. of from about 200 mOsm/kg to about 260 mOsm/kg, and comprises at least one relatively-ozone-inert buffering agent selected from the group consisting of boric acid, sodium tetraborate, potassium tetraborate, acetic acid, sodium acetate, potassium acetate, and a mixture thereof. The aqueous lens care solution is compatible with ozone electrolytically generated in an ozone-based lens care system as characterized by comprising about 30 mM or less of chloride ion and less than about 10 mM of one or more ozone-interfering buffering agents.

This invention relates generally to an aqueous lens care solution and a kit useful for cleaning and disinfecting a contact lens in an ozone-based lens care system.

### BACKGROUND OF THE INVENTION

Contact lenses provide a means for vision correction for a wide range of consumers. The advantages of contact lens wear are numerous. Improved convenience and improved appearance in comparison to spectacle glasses are probably the two most important advantages to most consumers. However, contact lenses require stringent care regimes in order to ensure comfort and avoid ocular infections. Proper care of contact lenses typically requires the consumer to periodically clean and disinfect the lenses, to prevent infection or other deleterious effects on ocular health which may be associated with contact lens wear.

One lens care system is the use of multiple-purpose solutions to clean, to disinfect, and to rinse contact lenses. These systems have been dominating most of the lens care market. Such popularity is most likely derived from the easiness and convenience provided by these new systems to consumers. In order to achieve a satisfactory disinfecting result, a contact lens has to be in a MPS solution for a sufficient time period. But, patients do not have a direct way to determine if their lenses have been in the lens care solution long enough to disinfect the lenses.

Another lens care system is the use of hydrogen peroxide solution as described in U.S. Pat. No. 4,585,488, U.S. Pat. No. 4,748,992, U.S. Pat. No. 4,812,173, U.S. Pat. No. 4,889,689, U.S. Pat. No. 4,899,914, U.S. Pat. No. 5,011,661, U.S. Pat. No. 5,275,784, U.S. Pat. No. 5,302,352, U.S. Pat. No. 5,468,448, U.S. Pat. No. 5,523,012, U.S. Pat. No. 5,196,174, U.S. Pat. No. 5,089,240, U.S. Pat. No. 5,558,846, U.S. Pat. No. 5,576,028, U.S. Pat. No. 5,609,264, U.S. Pat. No. 5,609,837, U.S. Pat. No. 5,756,044, U.S. Pat. No. 5,807,585, U.S. Pat. No. 5,958,351, U.S. Pat. No. 6,210,639, U.S. Pat. No. 6,440,411, U.S. Pat. No. 6,569,824, U.S. Pat. No. 6,945,389 and in copending U.S. patent application 61/261,844 filed 17 Nov. 2009 and 61/262,674 filed 19 Nov. 2009, herein incorporated by references in their entireties. However, one disadvantage is that the hydrogen peroxide in these systems must be neutralized/decomposed before lenses can safely be inserted to the ocular region by the patient.

A commonly-owned PCT patent application publication No. WO2008/021349 discloses a lens care system which comprises a colored lens care solution (a multipurpose solution or a hydrogen peroxide solution), a lens case having a singlet oxygen-generating agent covalently attached to the solution-contacting surface of the lens case, and a light source for gradually decomposing colorants in the colored lens care solution and rendering the colored lens care solution coloreless over a specific time period, thereby indicating that lenses under disinfecting and cleaning by the colored lens care solution are ready for use. Methods for disinfecting contact lenses disclosed in WO2008/021349 are still based on either multipurpose solutions or hydrogen peroxide solutions.

Another potential lens care system is utilization of ozone to disinfect contact lenses, as disclosed in WO9204098, WO09021936, US20120205255, U.S. Pat. No. 5,487,788, U.S. Pat. No. 5,129,999, and U.S. Pat. No. 5,082,558 (all of which are incorporated by reference in their entirety). While ozone can be an effective antimicrobial agent, its commercialization for contact lenses disinfection has been limited.

Thus, there is still a need for new lens care systems for disinfecting and cleaning contact lenses.

### SUMMARY OF THE INVENTION

Generally described, the present invention is related to an aqueous lens care solution for disinfecting and/or cleaning contact lenses, having an osmolality of from about 150 mOsm/kg (i.e., “milliosmoles per kilogram of water”) to about 260 mOsm/kg and a conductivity of from about 0.1 mS/cm to about 10.0 mS/cm, and comprising at least one relatively-ozone-inert buffering agent selected from the group consisting of boric acid, sodium tetraborate, potassium tetraborate, acetic acid, sodium acetate, potassium acetate, and a mixture thereof, wherein the aqueous lens care solution the aqueous lens care solution is compatible with ozone electrolytically generated in an ozone-based lens care system as characterized by comprising about 30 mM (i.e., “millimolar”) or less of chloride ion and less than about 10 mM of one or more ozone-interfering buffering agents.

The present invention is also related to a method or kit for disinfecting and/or cleaning contact lenses using an aqueous lens care solution of the invention.

The present invention provides the foregoing and other features, and the advantages of the invention will become further apparent from the following detailed description of the example embodiments set forth herein, read in conjunction with the accompanying figures. The detailed description and figures are merely illustrative of the invention and do not limit the scope of the invention, which is defined by the appended claims and equivalents thereof.

### BRIEF DESCRIPTION OF THE DRAWINGS

FIG. 1 shows the UV spectroscopy of phosphate buffered saline (PBS) ozone-treated for 5 minutes @ 150 mA (Right Insert: sodium hypochloride standard).

FIG. 2 shows the UV spectroscopy of phosphate buffer ozone-treated for 5 minutes @ 150 mA.

FIG. 3 shows the effects of age and exposure to ozone upon osmotic strength of test solutions.

FIG. 4 shows effects of ozone concentration upon citrate.

FIG. 5 shows comparison of dye degradation with borate and borate/sulfate buffer.

FIG. 6 shows the effect of pH on dye degradation.

FIG. 7 shows comparison of solution conductivity with the EOI conductance, measured by applying 4 V and measuring the resulting current.

### DETAILED DESCRIPTION OF EMBODIMENTS OF THE INVENTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art. Also, as used in the specification including the appended claims, reference to singular forms such as “a,” “an,” and “the” include the plural, and reference to a particular numerical value includes at least that particular value, unless the context clearly dictates otherwise. “About” as used herein means that a number referred to as “about” comprises the recited number plus or minus 1-10% of that recited number.

The present invention is generally directed to an aqueous lens care solution for use in an ozone-based lens care system for disinfecting and cleaning of contact lenses. Examples of such ozone-based lens care systems include those systems disclosed in US2012205255 (herein incorporated by reference in its entirety, QuickPure™ CONTACT LENS SANITIZER, or the like. The invention relies on ozone to disinfect and clean contact lenses. Ozone is a highly reactive species which has been used to deactivate bacteria, fungi, protozoa, and viruses, including the hard to kill oocyst-forming protozoa like Cryptosporidum parvum. It is discovered here that a high concentration of ozone can be generated electrolytically with relatively high stability in an ozone-based lens care system (e.g., one disclosed in US2012205255) by using an aqueous lens care solution of the invention and can be used to effectively disinfect, in some cases better than hydrogen peroxide solutions.

An aqueous lens care solution of the invention is compatible with ozone electrolytically generated in an ozone-based lens care system for ensure that high concentration of ozone can be generated electrolytically with relatively high stability in an ozone-based lens care system. The term “compatible with ozone” in reference to an aqueous lens care solution means that the aqueous lens care solution does not react significantly with ozone, which is generated during ozone-based disinfection/cleaning of contact lenses in an ozone-based lens care system (e.g., one of those described above), to form any toxic by-products in an amount sufficient to affect the corneal health of the eyes of a patient wearing the disinfected and cleaned contact lenses, and/or to reduce the availability of ozone for disinfecting and cleaning of contact lenses. (i.e., not to interfere with the disinfection and cleaning of contact lenses by ozone.

An aqueous lens care solution of the invention can be used in an ozone-based lens care system to disinfect and clean contact lenses including hard (PMMA) contact lenses, soft contact lenses, and rigid gas permeable (RGP) contact lenses. The soft contact lenses are hydrogel contact lenses or silicone hydrogel contact lenses.

For the purposes of the present invention the term “disinfect” means the rendering non-viable of substantially all pathogenic microbes that are in the vegetative state, including gram negative and gram positive bacteria, as well as fungi.

A “hydrogel” refers to a crosslinked polymeric material which can absorb at least 10 percent by weight of water when it is fully hydrated. Generally, a hydrogel material is obtained by polymerization or copolymerization of at least one hydrophilic monomer in the presence of or in the absence of additional monomers and/or macromers.

A “silicone hydrogel” refers to a hydrogel obtained by copolymerization of a polymerizable composition comprising at least one silicone-containing vinylic monomer or at least one silicone-containing macromer.

“Hydrophilic,” as used herein, describes a material or portion thereof that will more readily associate with water than with lipids.

In accordance with the invention, a lens care solution of the invention is ophthalmically safe. The term “ophthalmically safe” with respect to a lens care solution is meant that a contact lens treated with the solution is safe for direct placement on the eye without rinsing, that is, the solution is safe and sufficiently comfortable for daily contact with the eye via a contact lens. An ophthalmically safe solution has a tonicity and pH that is compatible with the eye and comprises materials, and amounts thereof, that are non-cytotoxic according to international ISO standards and U.S. FDA regulations.

The term “compatible with the eye” means a solution that may be in intimate contact with the eye for an extended period of time without significantly damaging the eye and without significant user discomfort.

It is found here that use of an aqueous lens care solution of the invention for disinfecting and cleaning contact lenses in an ozone-based lens care system neither adversely affects the lens mechanical properties and metrology, nor results in any significant, visual lens de-coloration. Unlike other oxidative systems (i.e. hydrogen peroxide) or multi-purpose solutions, ozone-based disinfection and cleaning of contact lenses by using an aqueous lens care solution of the invention can be a relatively short process, typically less than 1 hour for disinfection. Unlike other systems, this system eliminates chloride ions and prevents bleach formation. Additional heating beyond room temperature is not required, although the ozone generation can cause temperature increase to 45° C.

The invention, in one aspect, provides an aqueous lens care solution for cleaning and disinfecting contact lenses in an ozone-based lens care system. An aqueous lens care solution of the invention has an osmolality at about 25° C. of from about 150 mOsm/kg to about 260 mOsm/kg and comprises at least one relatively-ozone-inert buffering agent selected from the group consisting of boric acid, sodium tetraborate, potassium tetraborate, acetic acid, sodium acetate, potassium acetate, and a mixture thereof, wherein the aqueous lens care solution is compatible with ozone electrolytically generated in an ozone-based lens care system as characterized by comprising about 30 mM or less of chloride ion and less than about 10 mM of one or more ozone-interfering buffering agents.

An aqueous lens care solution of the invention is preferably formulated in such a way that it is hypotonic solution. The tonicity as measured by osmolality at 25° C. of the solution is typically adjusted to be in the range from about 150 to about 260 mOsm/kg, preferably from about 170 to about 250 mOsm/kg, more preferably from about 180 to about 240 mOsm/kg. Deviations from this concentration are possible throughout, provided that the contact lenses to be treated are not damaged. Further, an aqueous lens care solution of the invention is formulated to having a conductivity of from about 0.1 mS/cm to about 10 mS/cm, preferably from about 0.5 mS/cm to about 8 mS/cm, more preferably from about 0.5 mS/cm to about 6 mS/cm.

The concentration of chloride ions in an aqueous lens care solution of the invention is about 30.0 mM/L or less, preferably about 4.0 mM/L or less, more preferably about 2.0 mM/L or less, even more preferably about 0.05 mM/L or less, most preferably about 0.03 mM/L or less. It is also discovered here that although chloride salts are generally used in common lens care solutions to adjust osmotic strength, they are not suitable for ozone-based disinfection and cleaning of contact lenses, because ozone can react with the chloride ion to form undesirable by-products, such as, for example, hypochlorite (bleach), chlorine dioxide, or chlorine gas in solution. Production of these by-products during ozone-based disinfection and cleaning of contact lenses could adversely affect the cornea health (e.g., damaging the ocular region). Most preferably, an aqueous lens care solution of the invention is free of chloride ion.

An aqueous lens care solution of the invention is preferably formulated in such a way that it has a pH within a physiologically acceptable range of from about 5.5 to about −9.0, preferably from about 6.0 to about 8.0, more preferably from about 6.5 to about 7.0. The pH of the solution of the present invention preferably is maintained by at least one relatively-ozone-inert buffering agent selected from the group consisting of boric acid, sodium tetraborate, potassium tetraborate, acetic acid, sodium acetate, potassium acetate, and a mixture thereof. It is discovered here that boric acid, sodium borate (i.e., sodium tetraborate), potassium borate (i.e., potassium tetraborate), acetic acid, sodium perborate, potassium perborate, sodium acetate, and potassium acetate can react with ozone at a rate sufficiently slow so as not to significantly reduce the availability of ozone for disinfecting and cleaning contact lenses and are suitable for functioning as buffering agents in an aqueous lens care solution for disinfecting and cleaning of contact lenses in an ozone-based lens care system. In contrast to the above-list of relatively-ozone-inert buffering agents, other common buffering agents are ozone-interfering and not suitable for disinfecting and cleaning contact lenses in an ozone-based lens care system, because they can react with ozone at a rate sufficient fast to reduce significantly the availability of ozone for disinfecting and cleaning contact lenses. Examples of ozone-interfering buffering agents include phosphoric acid, phosphates (referring to Na₂HPO₄, NaH₂PO₄, Na₂HPO₄, KH₂PO₄, (NH₄)₂HPO₄, NH₄H₂PO₄, or a mixture thereof), citric acid, citrates (referring to potassium citrate, sodium citrate, ammonium citrate, or a mixture thereof), bicarbonates (referring to sodium bicarbonate, potassium bicarbonate, ammonium bicarbonate, or a mixture thereof), carbonates (referring to sodium carbonate, potassium carbonate, ammonium carbonate, or a mixture thereof), propionic acid, propionates (referring to potassium propionate, sodium propionate, ammonium propionate, or a mixture thereof) TRIS (2-amino-2-hydroxymethyl-1,3-propanediol) and salts thereof, Bis-Tris (Bis-(2-hydroxyethyl)-imino-tris-(hydroxymethyl)-methane) and salts thereof, bis-aminopolyols and salts thereof, triethanolamine and salts thereof, ACES (N-(2-hydroxyethyl)-2-aminoethanesulfonic acid) and salts thereof, BES (N,N-Bis(2-hydroxyethyl)-2-aminoethanesulfonic acid) and salts thereof, HEPES (4-(2-hydroxyethyl)-1-piperazineethanesulfonic acid) and salts thereof, MES (2-(N-morpholino)ethanesulfonic acid) and salts thereof, MOPS (3-[N-morpholino]-propanesulfonic acid) and salts thereof, PIPES (piperazine-N,N′-bis(2-ethanesulfonic acid) and salts thereof, TES (N-[Tris(hydroxymethyl)methyl]-2-aminoethanesulfonic acid) and salts thereof, and mixtures thereof.

In accordance with the invention, if one or more ozone reactive buffering agents are present in an aqueous lens care solution of the invention, they are present in an aqueous solution of the invention in an amount of 10 mM or less, preferably 5 mM or less, more preferably 2 mM or less, even more preferably 1 mM or less. Most preferably, an aqueous lens care solution of the invention is free of any ozone-interfering buffering agent.

In a preferred embodiment, an aqueous lens care solution of the invention further comprises an ozone-reactive dye. It is discovered here that an ozone-reactive dye can be used as an indicator for the completion of disinfecting and cleaning process. When an ozone-reactive dye present in an aqueous lens care solution of the invention is exposed to ozone, it reacts with the ozone, resulting in color change or disappearance. The complete visual de-coloration of the lens care solution at the end of each activated cycle, would indicate the completion of disinfection and cleaning of contact lenses. Examples of ozone-reactive dyes include FD&C Blue 1, D&C Green No. 5, FD&C Red No. 40, FD&C Yellow No. 5, and mixtures thereof. Those four dyes have adequate solubility, poor color fastness in oxidizers, and approval for use around the eye. Preferably, an aqueous lens care solution of the invention comprises FD&C Blue #1. Preferably, the ozone-reactive dye is present in an amount sufficient to ensure that the aqueous lens care solution becomes colorless under naked eyes over the time period of from about 3 to about 120 minutes, preferably from about 3 to about 90 minutes, more preferably from about 3 to about 60 minutes, even more preferably from about 3 to about 30 minutes, so as to provide a visual indicator for the completion of disinfection and cleaning of contact lenses in the ozone-based lens care system.

An aqueous lens care solution of the invention is preferably formulated in such a way that it has a viscosity of about 0.8centipoise to about 15 centipoises at 25° C., preferably from about 0.8 centipoises to about 10 centipoises at 25° C., more preferably from about 0.8 centipoises to about 1.1 centipoises at 25° C. It is known to a person skilled in the art how to adjust the viscosity of an aqueous solution by using one or more viscosity-enhancing agents.

In accordance with the invention, an aqueous lens care solution of the invention can further comprise from about 0.002% to about 0.5% by weight, more preferably from about 0.004% to about 0.1% by weight, even more preferably from about 0.005% to about 0.05% by weight of one or more components selected from the group consisting of lubricant(s), conditioning/wetting agent(s), tonicity agent(s), surfactant(s), chelating agent(s), defoaming agents, microbicide(s), preservative(s), and combinations thereof, based on the total amount of aqueous lens care solution.

A colored lens care solution of the invention preferably comprises a lubricant. “Lubricants” as used herein refer to any compounds or materials which can enhance surface wettability of a contact lens and/or the eye or reduce the frictional character of the contact lens surface. Examples of lubricants include without limitation mucin-like materials and hydrophilic polymers.

Exemplary mucin-like materials include without limitation polyglycolic acid, polylactides, collagen, and gelatin. A mucin-like material may be used to alleviate dry eye syndrome. The mucin-like material preferably is present in effective amounts.

Exemplary hydrophilic polymers include, but are not limited to, polyvinylalcohols (PVAs), polyamides, polyimides, polylactone, a homopolymer of a vinyl lactam, a copolymer of at least one vinyl lactam in the presence or in the absence of one or more hydrophilic vinylic comonomers, a homopolymer of acrylamide or methaacrylamide, a copolymer of acrylamide or methacrylamide with one or more hydrophilic vinylic monomers, mixtures thereof.

“Lubricants” as used herein refer to any compounds or materials which can enhance surface wettability of a contact lens and/or the eye or reduce the frictional character of the contact lens surface. Examples of lubricants include without limitation hydrophilic polymers.

An aqueous lens care solution of the invention can also comprise one or more conditioning/wetting agents (e.g., polyvinyl alcohol, polyoxamers, polyvinyl pyrrolidone, hydroxypropyl cellulose, and mixture thereof).

In accordance with the invention the aqueous lens care solution can further comprise a surfactant for cleaning the contact lens. Any suitable known surfactants can be used in the invention. Examples of suitable surfactants include, but are not limited to homopolymers of polyethylene glycol or polyethyleneoxide, poloxamers under the tradename Pluronic from BASF Corp. (Pluronic™ and Pluronic-R™) which are nonionic surfactants consisting of block copolymers of propylene oxide and ethylene oxide; poloxamine which is a block copolymer derivative of ethylene oxide and propylene oxide combined with ethylene diamine; tyloxapol, which is 4-(1,1,3,3-tetramethylbutyl)phenol polymer with formaldehyde and oxirane; ethoxylated alkyl phenols, such as various surface active agents available under the tradenames TRITON (Union Carbide, Tarrytown, N.Y., USA) and IGEPAL (Rhone-Poulenc, Cranbury, N.J., USA); polysorbates such as polysorbate 20, including the polysorbate surface active agents available under the tradename TWEEN (ICI Americas, Inc., Wilmington, Del., USA.); alkyl glucosides and polyglucosides such as products available under the tradename PLANTAREN (Henkel Corp., Hoboken, N.J., USA); and polyethoxylated castor oils commercially available from BASF under the trademark CREMAPHOR; and combinations thereof.

Preferred surfactants include polyoxypropylene-polyoxyethylene-polyoxypropylene block copolymers, poly(oxyethylene)-poly(oxybutylene) block copolymers disclosed in U.S. Pat. No. 8,318,144 (incorporated herein by reference in its entirety), certain poloxamers such as materials commercially available from BASF under the tradenames PLURONIC® surfactants, and combinations thereof. Examples of PLURONIC® surfactants include: PLURONIC® L42, PLURONIC® L43, and PLURONIC® L61. Examples of PLURONIC® R surfactants include: PLURONIC® 31R1, PLURONIC® 31R2, PLURONIC® 25R1, PLURONIC® 17R1, PLURONIC® 17R2, PLURONIC® 12R3, PLURONIC® 17R4, PLURONIC® F-68NF, PLURONIC® F68LF, and PLURONIC® F127. Examples of poly(oxyethylene)-poly(oxybutylene) block copolymers include di-block copolymer, denoted as PEO-PBO (i.e., polyoxyethylene-polyoxybutylene), a tri-block copolymer, represented as PEO-PBO-PEO or PBO-PEO-PBO, or other block-type configurations. When present, surfactants may be employed at a concentration of from about 0.005% to about 1% by weight, preferably from about 0.01% to about 0.5% by weight, more preferably from about 0.02% to about 0.25% by weight, even more preferably from about 0.04% to about 0.1% by weight, based on the total amount of aqueous lens care solution.

An aqueous lens care solution of the invention may include (but preferably does not include) an effective amount of a chelating agent. Any suitable, preferably ophthalmically acceptable, chelating agents may be included in the present compositions, although ethylenediaminetetraacetic acid (EDTA), salts thereof and mixtures thereof are particularly effective. EDTA is low level non-irritating chelating agent and can be synergistic with PHMB to increase antimicrobial efficacy. Typical amount of EDTA is from about 0.002% to about 0.5% by weight, more preferably from about 0.004% to about 0.1% by weight, even more preferably from about 0.005% to about 0.05% by weight, based on the total amount of aqueous lens care solution.

An aqueous lens care solution of the invention may include an antimicrobial agent in an amount effective to preserve the aqueous lens care solution. The term “an amount effective to preserve” means an amount of an antimicrobial agent effective in producing the desired effect of preserving the solutions described herein from microbial contamination, preferably an amount which, either singly or in combination with one or more additional antimicrobial agents, is sufficient to satisfy the preservative efficacy requirements of the United States Pharmacopoeia (“USP”). In a preferred embodiment, an aqueous lens care solution comprises about 100 ppm or less, preferably about 75 ppm or less, more preferably about 60 ppm or less, even more preferably about 50 ppm or less of a peroxide compound selected from the group consisting of hydrogel peroxide, sodium perborate tetrahydrate, sodium percarbonate, sodium persulfate, and combinations thereof.

An aqueous lens care solution of the invention is produced in known manner, in particular, by means of conventional mixing of the constituents with water or dissolving the constituents in water.

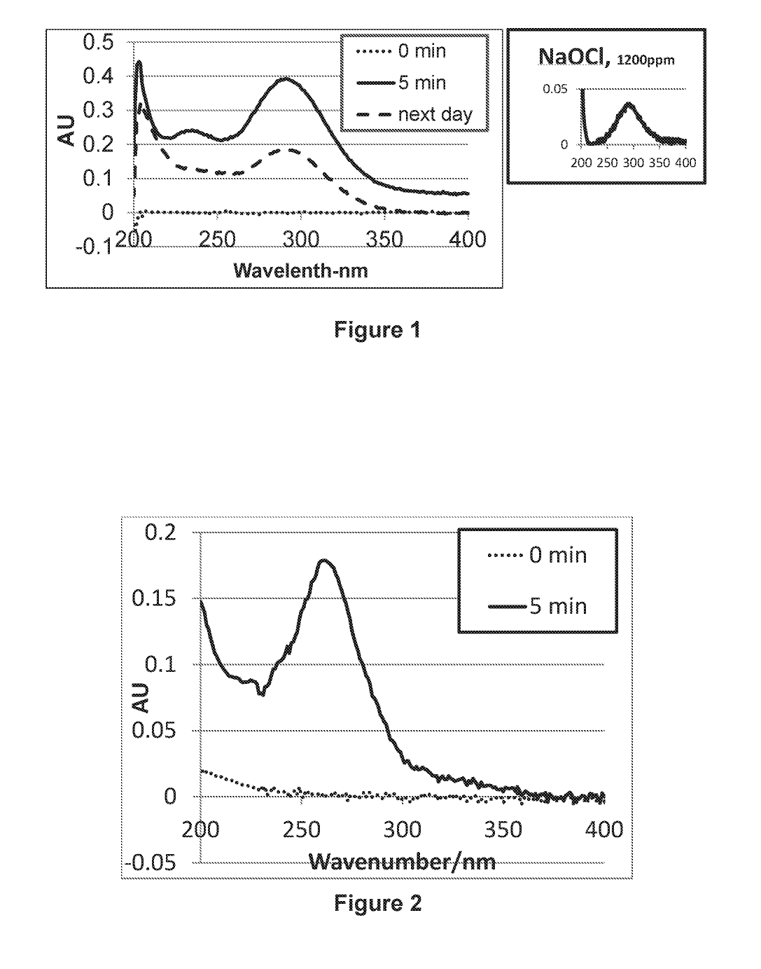

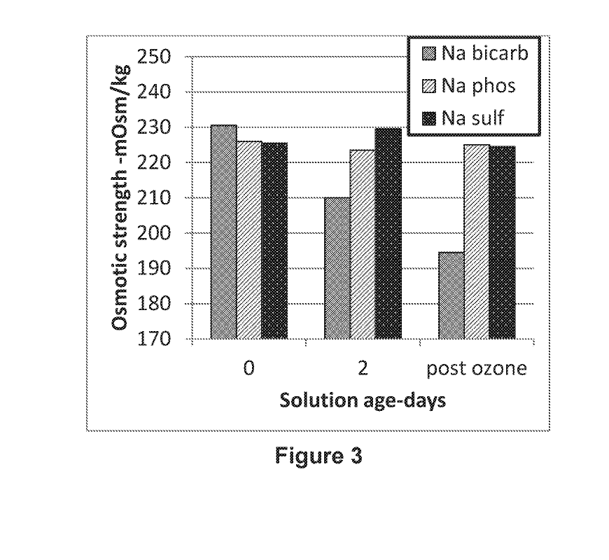

One or more contact lenses can be disinfected with an ozone-based lens care system, e.g., as illustrated in FIGS. 1A and 1B of US20120205255, by immersing the lens in an aqueous lens care solution (about 8 to 15 ml) of the invention in a contact lens disinfecting chamber (FIG. 3 of US20120205255) which is configured to hold one or more contact lenses. The concentration of ozone generated in the aqueous lens care solution of the invention is controlled in the range from about 2 to about 10 ppm. The time period is sufficient long for disinfecting contact lenses. It can be ranging from about 3 to about 120 minutes, preferably from about 3 to about 90 minutes, more preferably from about 3 to about 60 minutes, even more preferably from about 3 to about 30 minutes.

An aqueous lens care solution of the invention can be used in an ozone-based lens care system to disinfect contact lenses against a wide range of microorganisms including but not limited to Fusarium solani, Staphylococcus aureus, Pseudomonas aeruginosa, Serratia marcescens and Candida albicans.

In another embodiment, the invention provides a lens care kit for cleaning and/or disinfecting a contact lens, wherein the lens care kit comprises an aqueous lens care solution of the invention as described above.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments may be interchanged either in whole or in part or can be combined in any manner and/or used together. Therefore, the spirit and scope of the appended claims should not be limited to the description of the preferred versions contained therein.

The previous disclosure will enable one having ordinary skill in the art to practice the invention. In order to better enable the reader to understand specific embodiments and the advantages thereof, reference to the following non-limiting examples is suggested. However, the following examples should not be read to limit the scope of the invention.

### EXAMPLE 1

An aqueous lens care solution (formulation 1) is prepared to have the following composition: 80 mM boric acid (0.5% by weight); 1.4 mM borax (sodium tetraborate decahydrate) (0.052% by weight); 113 mM sodium chloride (0.66% by weight). Approximately 10 ml of the prepared solution is placed in an ozone-based lens care system similar to that (FIGS. 1A-1D) disclosed in US2012205255), to which a known number test microorganisms are added to the formulation. A Clear Care® cup is retrofitted into the ozone-based system of US2012205255 by cutting the bottom of the Clear Care® cup and is charged at 150 mA for different time durations. The results in Table 1 show good antimicrobial efficacy against Fusarium (within 30 seconds) and Acanthamoeba (within 3 minutes) microorganisms.

TABLE 1
log reduction of microorganisms

| | |
|---|---|
| | Acanthamoeba |
| Pulse Time | castellani |

| (0.15 Amps) | Fusarium solani | Trophs | Cysts |
|---|---|---|---|
| 30 seconds | 4.4 | 0.5 | 1.0 |
| 60 seconds | 4.4 | 2 | 1.3 |
| 180 seconds | 4.4 | 3.3 | 2.2 |

starting log count 5.4 for Fusarium, 4.3 log count for Acanthamoeba

Unfortunately, the solution during ozone-generation has a bleach-smell, indicating hypochlorite production. Colored test strips indicate more than 10 ppm hypochlorite is generated. Acuvue Oasys lenses treated for 60 seconds show no observable dye (color) bleaching at 50 mA, some dye (color) bleaching between 100 and 150 mA, and no color left between 200 and 250 mA. Alternatively, fixing current at 150 mA, no observable color change is seen at 30 seconds, some at 60 seconds, and little color left at 180 seconds. These data indicate that formation of hypochlorite does impact contact lens viability, and could impact the ocular surface and cause safety issues.

### EXAMPLE 2

This example illustrates the identification of the hyperchlorite production during ozone generation in presence of chloride species in the aqueous lens care solution. UV spectroscopy of ozone-reacted standard phosphate buffered saline (i.e., with NaCl, FIG. 1) shows a peak at 293, which is consistent with UV absorption of a hypochlorite standard (right insert). The hypochlorite peak is present even a day after reaction. Testing of a similar phosphate buffer without NaCl shows that the hypochloride peak at 293 nm is absent and a new peak at 263 nm appears (FIG. 2). This peak at 263 nm is consistent with what other investigators have been using to quantify ozone (see, E. J. Hart, K. Sehested, and J. Holoman, “Molar absorptivities of ultraviolet and visible bands of ozone in aqueous solutions,” Analytical Chemistry, vol. 55, no. 1, pp. 46-49, January 1983). This example illustrates that by removing the chloride ion from the formulation, hypochlorite formation during electrolysis can be eliminated.

### EXAMPLE 3

Aqueous lens care solutions are prepared to have the compositions shown in Table 2A without added chloride ions.

TABLE 2A

| Formulation | Concentration (mM) | | | | | | |
|---|---|---|---|---|---|---|---|
| No. | NaHPO₄ | Na₂PO4 | Na₂SO₄ | B(OH)₃ | Borax | Sodium citrate | NaHCO₃ |
| 3 | 0.83 | 187.4 | | | | | |
| 4 | 6.44 | 16.7 | | | | | |
| 5 | 6.44 | 16.7 | 70.4 | | | | |
| 6 | | | | 80.9 | 1.4 | | |
| 7 | 54.2 | | | 80.9 | 1.4 | | |
| 8 | | | | 80.9 | 1.4 | | 35.7 |
| 9 | | | 70.4 | 80.9 | 1.4 | | |
| 10 | | | | 80.9 | 1.4 | 14.01 | |
| 11 | | | | 80.9 | 1.4 | 70.06 | |

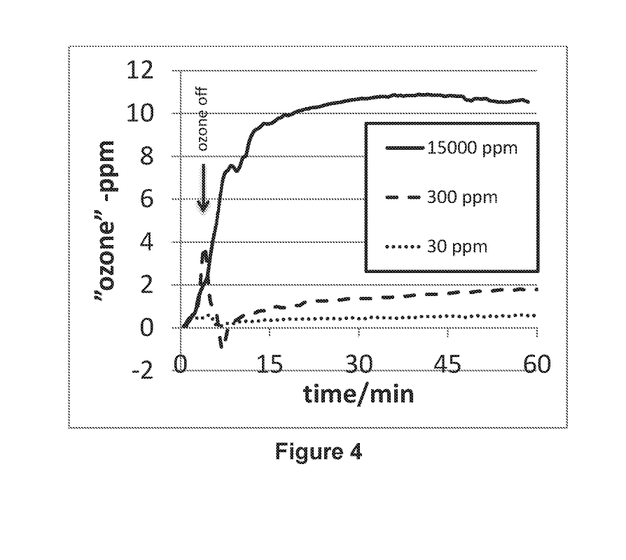

Most of the formulations (3-7, 9) show strong presence of ozone generation using the UV method as described previously (Table 2B). The borate/bicarbonate (formulation 8) shows good ozone generation, but the osmolarlity of the solution decreases with time and with exposure to ozone (see FIG. 3). The bicarbonate is thought to be degrading to form carbon dioxide. The borate/citrate solution (formulation 10-11) show a strong peak at 260 nm, where ozone has been identified previously. However, upon further analysis, the peak height at 260 nm is a function of both ozone and citrate concentration (FIG. 4). The citrate, a known antioxidant, could be reacting forming a complex with ozone. The citrate could interfere with ozone's effectiveness to provide disinfection.

TABLE 2B

| | Properties | | |
|---|---|---|---|
| Formulation No. | pH | Osmolality | Observation |
| 3 | 9 | | good ozone detection |
| 4 | | 49 | good ozone detection |
| 5 | | 215 | trace ozone, lots of bubbles |
| 6 | 7 | 85 | Repeatable ozone detection |
| 7 | | 189 | ozone for 3 min, then bubbles |
| 8 | | 212 | ozone for 4 min, then bubbles |
| 9 | | 250 | trace ozone, lots of bubbles |
| 10 | | 107 | high (>4 ppm) ozone, 25% decay |
| | | | in 10 hr |
| 11 | | 241 | high (>10 ppm) ozone, no decay |

### EXAMPLE 4

This example illustrates a method for determining whether a buffer solution can provide the best ozone activity.

### Preparation of Aqueous Lens Care Solutions

Aqueous lens care solutions are prepared as follows.
Formulation 12 (112 mM acetate, Osmolarlity: 219 mOsm/kg): 0.004 g acetic acid; 0.921 g sodium acetate; and 100 g water.
Formulation 13 (116 mM Na propionate, Osmolarlity: 219 mOsm/kg): 1.12 g sodium propionate; and 100 g water.
Formulation 14 (220 mM TRIZMA (primary amine), Osmolarlity: 211 mOsm/kg): 2.53 g TRIZMA® (from Sigma); and 97.47 g water.
Formulation 15 (132 mM HEPES (piperazine system), Osmolarlity: 218 mOsm/kg): 2.82 g TRIZMA® (from Sigma); and 97.18 g water.
Formulation 16 (Osmolarlity: 225 mOsm/kg): 0.77 g malonic acid disodium; and 50 g water.
Formulation 17 (Osmolarlity: 240 mOsm/kg): 1.27 g disodium succinate; and 50 g water.
Formulation 18 (Osmolarlity: 208 mOsm/kg): 0.71 g glutaric acid, sodium; and 50 g water.
Formulation 19 (pH 6; Osmolarlity: 223 mOsm/kg): 13.86 g boric acid; 0.120 g Borax; and 1000 g water.
Formulation 20 (pH 7; Osmolarlity: 225 mOsm/kg): 13.64 g boric acid; 1.364 g Borax; and 1000 g water.
Formulation 21 (pH 8; Osmolarlity: 231 mOsm/kg): 11.79 g boric acid; 8.065 g Borax; and 1000 g water.
Formulation 22 (pH 6; Osmolarlity: 214 mOsm/kg): 5.431 g sodium acetate; 10.7 g acetic acid; 5.01 g boric acid; 0.04 g Borax; and 995 g water.
Formulation 23 (pH 7; Osmolarlity: 212 mOsm/kg): 5.431 g sodium acetate; 2.75 g acetic acid; 4.93 g boric acid; 0.49 g Borax; and 995 g water.
Formulation 24 (pH 8; Osmolarlity: 206 mOsm/kg): 5.431 g sodium acetate; 2.97 g boric acid; 2.03 g Borax; and 995 g water.
Formulation 25 (pH 6; Osmolarlity: 207 mOsm/kg): 8.04 g NaHPO₄; 2.13 g Na₂PO₄; 5.01 g boric acid; 0.04 g Borax; and 1040 g water.
Formulation 26 (pH 7; Osmolarlity: 208 mOsm/kg): 1.86 g NaHPO₄; 7.12 g Na₂PO₄; 4.93 g boric acid; 0.49 g Borax; and 1135 g water.
Formulation 27 (pH 8; Osmolarlity: 206 mOsm/kg): 0.25 g NaHPO₄; 9.68 g Na₂PO₄; 2.97 g boric acid; 2.03 g Borax; and 1052 g water.
Formulation 28 (1.7 g sodium phosphate monobasic monohydrate, 100 ml water, Osmolarity 222 mOsm)
Formulation 29 (1.472 g sodium sulfate, 100 g water, 237 mOsm)
Formulation 30 (0.883 g sodium sulfate, 0.5 g boric acid, 0.05 borax, 231 mOsm)
Formulation 31 (0.676 g sodium bicarbonate, 0.5 g boric acid, 0.05 borax, 226 mOsm)
Formulation 32 (1.0 g potassium sulfate, 0.5 g boric acid, 0.05 borax, 220 mOsm)
Formulation 33 (1.6 potassium sulfate monohydrate, 0.5 g boric acid, 0.05 borax, 224 mOsm)

### Testing of Dye Degradation by Ozone Generated in the Aqueous Lens Care Solutions

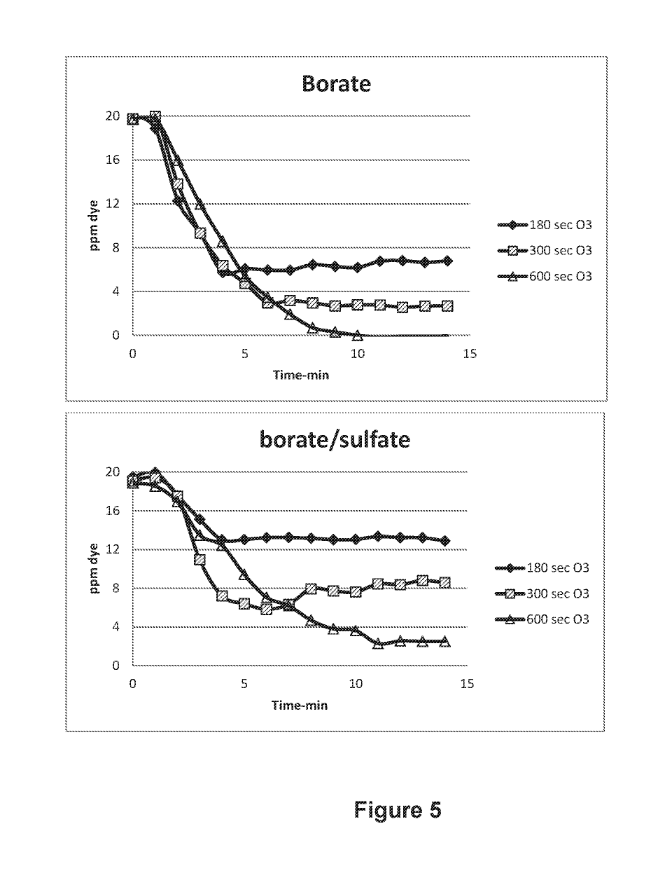

A 20 ppm FD&C Blue #1 dye is added to different buffer solutions, although other dyes could be used (i.e. FD&C Red 40, FD&C yellow 5, FD&C green #5). The dye can be monitored using visible light absorption during ozone exposure using a UV probe. The more rapid that dye can be removed for a certain amount of time will provide an indication of ozone activity. For example, the dye degradation for a borate solution (Formulation 20, see FIG. 5. Top) at three different ozone exposure times can be compared with a borate/sulfate solution with added dye. The dye rate degradation constant for each of these solutions are calculated by determining how much dye degrades. For example, in FIG. 5, the borate solution has reduced more dye than the borate/sulfate after 180 sec ozone treatment.

A number of different solutions prepared above are tested, and their dye degradation amount are measured after 300 seconds ozone production (150 mA) (see Table 3). This data allows the solutions having the most active ozone to be ranked.

TABLE 3

| | | | Dye | |
|---|---|---|---|---|
| Formu- | | | degradation | |
| lation | | | (20 ppm | % Dye |
| ID | Description | Osmolarity | start) | degradation |
| | DI water | 0 | 17.1 | 97% |
| 23 | Borate/acetate | 205 | 16.6, 16.2 | 98%, 93% |
| 12 | Acetate | 219 | 16.7 | 88% |
| 20 | borate buffer | 220 | 17.1 | 86% |
| 14 | Triz MA | 219 | 13.0 | 66% |
| 16 | Malonate | 225 | 11.0 | 64% |
| 18 | glutarate | 208 | 10.9 | 64% |
| 26 | borate/sodium | 226 | 12.1 | 64% |
| | phosphate | | | |
| 28 | Na phosphate | 222 | 11.6 | 61% |
| | mono basic | | | |
| 29 | Na sulphate | 237 | 11.6 | 60% |
| 30 | borate/sodium | 231 | 10.5 | 55% |
| | sulfate | | | |
| 31 | Borate/sodium | 226 | 10.1 | 54% |
| | bicarbonate | | | |
| 32 | Borate/K | 220 | 8.7 | 46% |
| | Sulphate | | | |
| 33 | Borate/potassium | 224 | 7.9 | 41% |
| | phosphate | | | |
| 13 | Sodium | 218 | 5.2 | 28% |
| | propionate | | | |
| 17 | succinate | 240 | 4.1 | 25% |
| 15 | HEPES | 218 | 1.0 | 5% |

### EXAMPLE 5

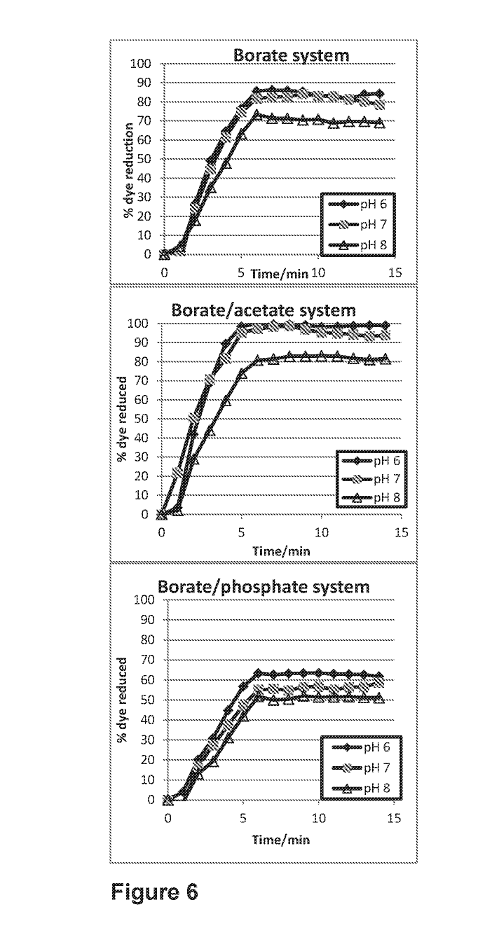

The effect of pH on dye degradation is tested, using the formulations prepared in Example 4. As shown in FIG. 6, the borate and borate acetate formulations degrade dye better than the borate/phosphate system, as expected from Table 3. pH does seem to have an influence, with lower pH performing better than higher pH.

### EXAMPLE 6

Solution 20 and 23 prepared in Example 4 are used to test microbial efficacy. Table 4 shows the log reduction of Fusarium Solani and Acanthamoeba cyst. Both solutions are efficient in achieving essentially complete kill of organisms at 60 minutes. Ninety percent of Acanthamoeba cysts (1 log) are achieved in only 5-15 minutes for either solution.

TABLE 4
Log reduction of microorganisms after ozone treatment. Initial count
for Fusarium is 1.7 × 10⁵, Acanthamoeba is 3.7 × 10⁴ organisms.
A log reduction of 4 indicates no detectible organisms remaining.

| | | Solution | | |
|---|---|---|---|---|
| | Solution 13-11, | 14-2, | Solution 13-11 | Solution 14-2 |
| | Fusarium, | Fusarium | Acanthameoba | Acanthamoeba |
| Condition | Solani | Solani | Cysts | Cyst |
| 0 min | 0.1 | −0.1 | 0.1 | 0.1 |
| 5 min | 1.7 | 1.1 | 1.0 | N/A |
| 15 min | 2.0 | 1.8 | 1.1 | 1.1 |
| 60 min | 4.4 | 4.3 | 1.1 | 0.9 |

### EXAMPLE 7

In the presence of organic material, such as albumin, solutions that are ozone treated sometimes can show foaming. New formulations (based upon formulation 23) are made with different amounts of typical PPO-PEO surfactants (Table 5) to determine if foaming could be controlled, yet have minimal impact with ozone production. Foaming is determined by placing a 2 foot long extension onto the ozone producing cup, and measuring how high the foam extends into the tube (Table 6—Foam height of solution, after addition of 6 ppm of albumin to ozone formulation). The starting solution height is 25 mm (0 mm foam height). The best surfactants for deforming this solution is 17R4 (500 ppm) and 31R1 (10 ppm).

The bovine serum albumin (BSA, 6 ppm) control shows foam heights of close to 30 mm in only 5 minutes. Some surfactants (i.e. 17R4) are found to be sufficiently surface active below its CMC to stop foaming, while others (31R1) show good foam control over its CMC.

Similar to the technique described in Example 6, the time required to degrade the dye to 50% of its original value can be used to rate ozone activity. These formulations show good dye degradation results (see Table 7).

Other surfactants could be added as defoamers, comfort agents, surface wetting, or protein/lipid removal agents.

TABLE 5

| | weight-g | | | | | weight-g | | |
|---|---|---|---|---|---|---|---|---|
| Formulation | | | sodium | 0.5M | | FD&C | Pluronic | Pluronic |
| ID | H₃BO₄ | Na₂B₄O₇—10H₂O | acetate | acetic acid | water | blue #1, | 17R4 | 31R1 |
| 28 | 5.3 | 0.5 | 5.59 | 1.5 | 1000 | 0.01 | | |
| 9 | 5.081 | 0.019 | 5.431 | 1.281 | 1000 | 0.01 | | |
| 30 | 5.081 | 0.019 | 5.431 | 1.281 | 1000 | 0.01 | 0.50 | |
| 31 | 5.081 | 0.019 | 5.431 | 1.281 | 1000 | 0.01 | | 0.01 |
| 32 | 5.081 | 0.019 | 5.431 | 1.281 | 1000 | | | |
| 35 | 13.64 | 1.364 | | | 1000 | 0.01 | | |

TABLE 6

| | | | Foam height |
|---|---|---|---|
| Surfactant | CMC (ppm) | Concentration. (ppm) | Mm @ 5 min |
| BSA control | — | — | 29 |
| P103 | 50 | 100 | 22 |
| 17R4 | 91000 | 50 | 26 |
| 17R4 | | 500 | 8 |
| 31R1 | 7.1 | 5 | 15 |
| 31R1 | | 10 | 7 |

TABLE 7

| Formulation ID | Formulation ID | t ½ (average) |
|---|---|---|
| 28 | control-pH 7, 55A, | 2.8 |
| 29 | control-pH 6.5, 55B | 1.6 |
| 30 | 17R4-500 ppm, 55D | 1.2 |
| 31 | 31R1-10 ppm, 55C | 1.4 |

### EXAMPLE 8

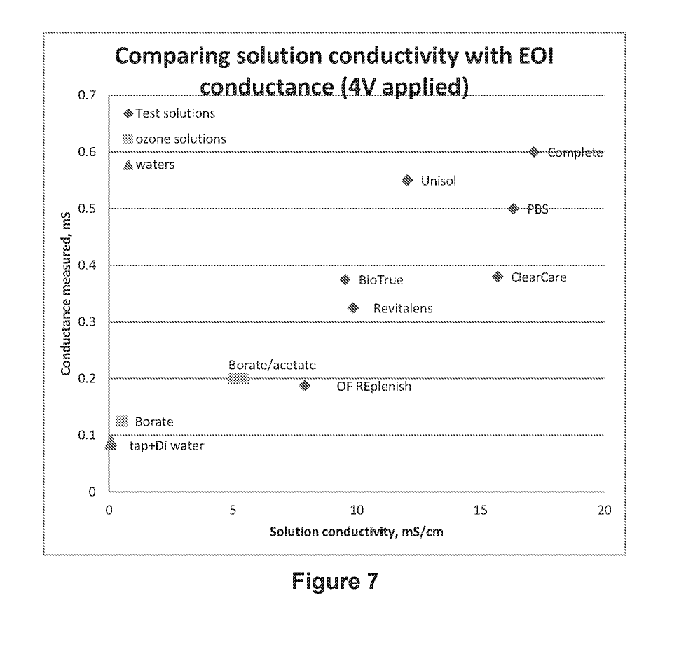

One concern with these solutions is a consumer using the wrong solution in the ozone generating device. For example, if a consumer accidentally put in phosphate buffered saline (PBS), the unit could produce hypochlorite species, which could affect ocular comfort and safety. As a fail-safe mechanism, the base unit could detect the solution conductivity and only turn on if the proper conductivity range is measured. As shown in Table 8, most conventional contact solutions are highly conductive (8-17 mS/cm), while tap or deionized water is low (<0.1 mS/cm). If the ozone buffers can be specifically tailored to be within a range of 0.2-7 mS/cm, then the failsafe mechanism could be utilized. To test the capability of the ozone producing unit to measure the conductivity of different solutions, a known voltage (4 V in this example) is applied, and the resulting current is measured. A current is measured between 0.5 and 0.8 mA would indicate that a correct solution has been added, while a current either >0.75V or <0.4 V would be rejected by the device. A plot comparing the solution conductivity and the calculated device conductance measured is shown in FIG. 7.

TABLE 8

| Formulation | Solution | EOI | |
|---|---|---|---|
| ID/commercial | Conductivity, | current, mA, | EOI conductivity, |
| product | mS/cm | @ 4 V | mS |
| Optifree Replenish | 7.9 | 0.75 | 0.1875 |
| Clear Care | 15.69 | 1.52 | 0.38 |
| Unisol 4 | 12.04 | 2.2 | 0.55 |
| Complete | 17.16 | 2.4 | 0.6 |
| Phosphate buffered | 16.33 | 2 | 0.5 |
| saline | | | |
| BioTrue | 9.54 | 1.5 | 0.375 |
| Revitalens | 9.85 | 1.3 | 0.325 |
| 20 | 0.5 | 0.5 | 0.125 |
| 31 | 0.51 | 0.5 | 0.125 |
| 23 | 5.43 | 0.8 | 0.2 |
| 28 | 5.02 | 0.8 | 0.2 |
| 29 | 3.4 | | |
| Deionied water | 0.037 | 0.34 | 0.085 |
| tap water | 0.085 | 0.36 | 0.09 |

### EXAMPLE 9

The solution viscosities of various formulations are tested with a Brookfield viscometer, (model DVII+ Pro). Table 9 provides the viscosities at different shear rates. All are within 0.9-10. Formulation 33 is composed of 0.07 mM monosodium phosphate (monohydrate) and 10 mM disodium phosphate (heptahydrate), while Formulation 34 is composed of 0.7 mM monosodium phosphate (monohydrate) and 99 mM disodium phosphate (heptahydrate).

TABLE 9
Viscosity of typical formulations, as
measured by the Brookfield viscometer

| | Viscosity (cps) | | |
|---|---|---|---|
| Formulation | 3 RPM | 6 RPM | 12 RPM |
| 33 | 0.92 | 0.92 | 0.92 |
| 34 | 1.02 | 0.97-1.02 | 1.05 |
| 23 | 0.97-1.02 | 1.02 | 1.02 |
| 32 | 1.02 | 0.97 | 0.95 |
| 30 | 0.92 | 0.92-0.97 | 0.92-0.95 |

What is claimed is:

1. An aqueous lens care solution for disinfecting and/or cleaning contact lenses in an ozone-based lens care system,
having an osmolality at about 25° C. of from about 150 to about 260 mOsm/kg, preferably from about 170 to about 250 mOsm/kg, more preferably from about 180 to about 240 mOsm/kg, and a conductivity of from about 0.1 mS/cm to about 10 mS/cm, preferably from about 0.5 mS/cm to about 8 mS/cm, more preferably from about 0.5 mS/cm to about 6 mS/cm; and
comprising at least one relatively-ozone-inert buffering agent selected from the group consisting of boric acid, sodium tetraborate, potassium tetraborate, acetic acid, sodium acetate, potassium acetate, and a mixture thereof,
wherein the aqueous lens care solution is compatible with ozone electrolytically generated in an ozone-based lens care system as characterized by comprising (1) about 30.0 mM/L or less, preferably about 4.0 mM/L or less, more preferably about 2.0 mM/L or less, even more preferably about 0.05 mM/L or less, most preferably about 0.03 mM/L or less of chloride ions and (2) about 10 mM or less, preferably 5 mM or less, more preferably 2 mM or less, even more preferably 1 mM or less of one or more ozone-interfering buffering agents.

2. The aqueous lens care solution of claim 1, wherein the aqueous lens care solution has a pH of from about 5.5 to about −9.0, preferably from about 6.0 to about 8.0, more preferably from about 6.5 to about 7.0.

3. The aqueous lens care solution of claim 1 or 2, wherein the one or more ozone-interfering buffering agents are selected from the group consisting of phosphoric acid, phosphates (referring to Na₂HPO₄, NaH₂PO₄, Na₂HPO₄, KH₂PO₄, (NH₄)₂HPO₄, NH₄H₂PO₄, or a mixture thereof), citric acid, citrates (referring to potassium citrate, sodium citrate, ammonium citrate, or a mixture thereof), bicarbonates (referring to sodium bicarbonate, potassium bicarbonate, ammonium bicarbonate, or a mixture thereof), carbonates (referring to sodium carbonate, potassium carbonate, ammonium carbonate, or a mixture thereof), propionic acid, propionates (referring to potassium propionate, sodium propionate, ammonium propionate, or a mixture thereof) TRIS (2-amino-2-hydroxymethyl-1,3-propanediol) and salts thereof, Bis-Tris (Bis-(2-hydroxyethyl)-imino-tris-(hydroxymethyl)-methane) and salts thereof, bis-aminopolyols and salts thereof, triethanolamine and salts thereof, ACES (N-(2-hydroxyethyl)-2-aminoethanesulfonic acid) and salts thereof, BES (N,N-Bis(2-hydroxyethyl)-2-aminoethanesulfonic acid) and salts thereof, HEPES (4-(2-hydroxyethyl)-1-piperazineethanesulfonic acid) and salts thereof, MES (2-(N-morpholino)ethanesulfonic acid) and salts thereof, MOPS (3-[N-morpholino]-propanesulfonic acid) and salts thereof, PIPES (piperazine-N,N′-bis(2-ethanesulfonic acid) and salts thereof, TES (N-[Tris(hydroxymethyl)methyl]-2-aminoethanesulfonic acid) and salts thereof, and mixtures thereof.

4. The aqueous lens care solution of claim 1, 2, or 3, wherein the aqueous lens care solution further comprises an ozone-reactive dye.

5. The aqueous lens care solution of claim 4, wherein the ozone-reactive dye is selected from the group consisting of FD&C Blue 1, D&C Green No. 5, FD&C Red No. 40, FD&C Yellow No. 5, and mixtures thereof.

6. The aqueous lens care solution of claim 5, wherein the ozone-reactive dye is FD&C Blue 1.

7. The aqueous lens care solution of claim 5 or 6, wherein the ozone-reactive dye is present in an amount sufficient to ensure that the aqueous lens care solution becomes colorless under naked eyes over the time period of from about 3 to about 120 minutes, preferably from about 3 to about 90 minutes, more preferably from about 3 to about 60 minutes, even more preferably from about 3 to about 30 minutes, so as to provide a visual indicator for the completion of disinfection and cleaning of contact lenses in the ozone-based lens care system.

8. The aqueous lens care solution of any one of claims 1 to 7, wherein the aqueous lens care solution has a viscosity of about 0.8 to about 15 centipoises at 25° C., preferably from about 0.8 to about 10 centipoises at 25° C., more preferably from about 0.9 to about 1.1 centipoises at 25° C.

9. The aqueous lens care solution of any one of claims 1 to 8, wherein the aqueous lens care solution comprises an antimicrobial agent in an amount effective to preserve the aqueous lens care solution.

10. The aqueous lens care solution of claim 9, wherein the antimicrobial agent is selected from the group consisting of hydrogen peroxide, sodium perborate tetrahydrate, sodium percarbonate, sodium persulfate, and combinations thereof.

11. The aqueous lens care solution of any one of claims 1 to 10, wherein the aqueous lens care solution is formulated in a way that the concentration of ozone generated in the aqueous lens care solution of the invention is controlled in the range from about 0.2 to about 10 ppm.

12. The aqueous lens care solution of any one of claims 1 to 11, wherein the aqueous lens care solution comprises from about 0.005% to about 1% by weight, preferably from about 0.01% to about 0.5% by weight, more preferably from about 0.02% to about 0.25% by weight, even more preferably from about 0.04% to about 0.1% by weight of a surfactant.

13. The aqueous lens care solution of claim 12, wherein the surfactant is selected from the group consisting of polyoxypropylene-polyoxyethylene-polyoxypropylene tri-block copolymers, poly(oxyethylene)-poly(oxybutylene) di-block copolymer, polyoxyethylene-polyoxybutylene-polyoxyethylene tri-block copolymer, polyoxybutylene-polyoxyethylene-polybutylene tri-block copolymer, and combinations thereof.

14. The aqueous lens care solution of any one of claims 1 to 13, wherein the aqueous lens care solution further comprises from about 0.002% to about 0.5% by weight, more preferably from about 0.004% to about 0.1% by weight, even more preferably from about 0.005% to about 0.05% by weight of one or more components selected from the group consisting of lubricant(s), conditioning/wetting agent(s), tonicity agent(s), chelating agent(s), defoaming agents, microbicide(s), and combinations thereof.

15. A lens care kit, comprising a lens care solution of any one of claims 1 to 14.
